(19) United States
(12) Patent Application Publication
(10) Pub. No.: US 2005/0287193 A1
(43) Pub. Date: Dec. 29, 2005

(54) Wound dressing made of chitin and alginate and method for forming the same

(75) Inventor: Jui-Sheng Lee, San Chung City (TW)

Correspondence Address: THE WEBB LAW FIRM, P.C., 700 KOPPERS BUILDING, 436 SEVENTH AVENUE, PITTSBURGH, PA, 15219, US

(73) Assignee: Taiwan Textile Research Institute

(21) Appl. No.: 11/072,051

(22) Filed: Mar. 4, 2005

(30) Foreign Application Priority Data
Jun. 29, 2004 (TW) 93119129

(51) Int. Cl. A61L 15/00

(52) U.S. Cl. 424/445; 514/55

(57) ABSTRACT

A sponge wound dressing made from chitin and alginate and a method for forming the same are described. Chitin and alginate are mixed and then dried to form a sponge. After immersing the sponge into a solution of alkaline earth metal salt, the alkaline earth metal alginate is formed. An optional cleaning step is followed to form a sponge wound dressing made of chitin and alginate.

### RELATED APPLICATIONS

The present application is based on, and claims priority from, Taiwan Application Serial Number 93119129, filed on Jun. 29, 2004, the disclosure of which is hereby incorporated by reference herein in its entirety.

### BACKGROUND

1. Field of Invention

The present invention relates to wound dressings and a method for forming the same. More particularly, the present invention relates to wound dressings made of chitin and alginate.

2. Description of Related Art

Keeping wounds moist is beneficial for wound healing. Additionally, several growth factors in wound exudates are good for healing. Therefore, various kinds of wound dressings are being developed, such as PU film, chitin and chitosan composite wound dressings and wound dressings made of collagen or alginate. Wound dressings are conventionally made in the form of a sponge, film or non-woven material, such as Kaltostat (Britcair Co.), a commercial non-woven wound dressing.

Alginic acid extracted from alga is a natural polymer formed of β -D-mannuronic acid and α-L-guluronic acid by polymerization. Alkaline earth metal alginate, such as calcium alginate, promotes hemostasis and provides wound protection. Therefore, alginate is applied in the clinical treatment of wounds, such as, for example, gashes, bums, sores and split-thickness skin graft donor sites.

One of conventional processes for forming a sponge wound dressing comprises the steps of preparing 5% sodium alginate aqueous solution; forming a sponge and immersing the sponge in a calcium chloride (CaCl₂) solution for solidification after freeze-drying the sodium alginate aqueous solution. Another conventional method comprises the following steps. A water-soluble polymer is added into the alginate solution after preparing a sodium alginate aqueous solution. The alginate solution is molded into a membrane and then the membrane is immersed in the calcium chloride solution for solidification. The membrane is boiled in water to melt away the water-soluble polymer and results in a porous wound dressing (Japan patent H7-179649, S62-250040). Still another method for forming a wound dressing comprises steps described as follows. A sodium alginate solution is prepared. A cross-linking reagent such as polyethyleneimine or carbodiimine is added to the alginate solution. A sponge is formed by freeze-drying the solution. The sponge is immersed in a calcium chloride solution for solidification and then dried (J. Biomed Mater. Res. 1999; 48(4), 522-527, “In vivo evaluation of a Novel Alginate dressing”).

Chitin, a wound dressing material extracted from crab, shrimp shells, insects and cuttlefish cartilage, is β-1,4-linked aminopolysaccharide of N-acetyl-D-glucosamine, and has an excellent biocompatibility to mammalian cells and anti-bacterial effect. According to the stereo structure, there are three chitins, α-chitin, β-chitin and γ-chitin. Due to the stability of the stereo structure between the molecules, β-chitin can absorb liquid, and can be formed in short staple by blending at high speed. Recent publications show that chitin has been used as a wound-healing material and is good for wound healing. (Japan paten S61-64256 and 3-41131).

There are many conventional wound dressings, but suffer from drawbacks. For example, the excess calcium ions released from conventional calcium alginate wound dressings may cause cell toxicity. Further, conventional chitin wound dressings (JP 3-41131) have low tensile strength and are breakable after absorbing wound exudates and thus inconvenient to use.

### SUMMARY

It is therefore an aspect of the present invention to provide a wound dressing that is anti-bacterial, absorbs exudates, allows gas to permeate therethrough, and promotes hemostasis and healing characteristics.

It is another an aspect of the present invention to provide a wound dressing with tensile strength and flexibility and that does not adhere to the wound or break.

It is still another aspect of the present invention to provide a method of producing a wound dressing that improves conventional methods of producing a sponge wound dressing by cross-linking agents or other chemical polymers.

In accordance with the foregoing and other aspects of the present invention, a wound dressing made of chitin and alginate is provided, which absorbs wound exudates, promotes hemostasis, is gas-permeable and stimulates healing. The wound dressing of the present invention also provides proper tensile strength and flexibility in use and is easy to apply to wounds.

In accordance with the foregoing and other aspects of the present invention, a method for producing the wound dressing of the invention is achieved. Chitin and an alkali metal alginate are mixed in a specific ratio to form a solution. The chitin is, for example, β-chitin, and the alkali metal alginate is, for example, sodium alginate or potassium alginate. The solution is then stirred at a high speed, such as 12000 rpm, to transform the chitin into a short staple form. The solution is freeze-dried to result in a sponge. Then, the sponge is immersed in an alkaline earth metal salt solution, such as calcium chloride (CaCl₂). After washing the sponge with water and deionized water several times, the wound dressing of the present invention is obtained by a drying process such as vacuum drying.

The ratio of alkali metal alginate to β-chitin is preferably between about 1:9 and 9:1 by weight (w:w), and between about 1:4 and 1:2 (w:w). The alkali metal salts may be beryllium salts, magnesium salts, calcium salts, strontium salts, barium salts or any combination thereof.

It is to be understood that both the foregoing general description and the following detailed description are by examples, and are intended to provide further explanation of the invention as claimed.

### BRIEF DESCRIPTION OF THE DRAWINGS

The invention can be more fully understood by reading the following detailed description of the preferred embodiment, with reference made to the accompanying drawings as follows:

FIG. 1 is a flowchart explaining the procedures for forming a wound dressing of the present invention;

FIG. 2 is a SEM micrograph showing the surface and cross section of the wound dressing according to one preferred embodiment of the present invention;

FIG. 3 is a line graph explaining the relationship between the ratio of alginate and chitin, tensile strength and liquid absorption ability;

FIG. 4 is a line graph showing the wound-repairing comparison of the present invention and other wound dressings.

### DESCRIPTION OF THE PREFERRED EMBODIMENTS

Reference will now be made in detail to the present preferred embodiments of the invention, examples of which are illustrated in the accompanying drawings. Wherever possible, the same reference numbers are used in the drawings and the description to refer to the same or like parts. All the preferred embodiments are described to make the features and the preparation method of the invention clear.

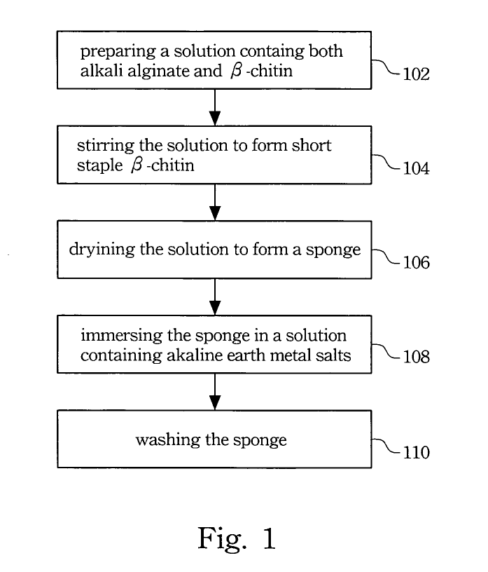

FIG. 1 is a flow chart illustrating steps of a preparation method in accordance with one example of the invention. In the first step102, chitin and an alkali metal alginate are mixed in a specific ratio and dissolved with water to form a solution. The chitin may be β-chitin, and the alkali metal alginate are, for example, sodium alginate or potassium alginate. The ratio of alkali metal alginate to chitin is, for example, from about 1:9 to 9:1 (w:w).

In second step (104), the solution is stirred at high speed, such as 12000 rpm or higher. After stirring, a hydrogel containing short staple chitin is obtained. In the third step (106), the hydrogel is dried to form a sponge by freeze-drying.

The fourth step (108) is to immerse the sponge in an alkaline earth metal salt solution, such as 5% calcium chloride solution. In the fourth step (108), the alkali metal alginate of the sponge reacts with the alkaline metal ions, such as calcium ions (Ca²⁺), to form insoluble alginate films, such as calcium alginate films. Then, the wound dressing of one preferred embodiment of the present invention is obtained. Optionally, the wound dressing can be washed with water and deionized water (D.I. water) several times to remove excess salts in the wound dressing.

In the foregoing steps, films and fabrics, such as polyethylene (PE), polypropylene (PP), poly(ethylene terephthalate) (PET), nylon, polyurethane (PU), cotton, wool and other natural or synthetic fabrics, can be used as a supporter in the wound dressing or on a side of the wound dressing.

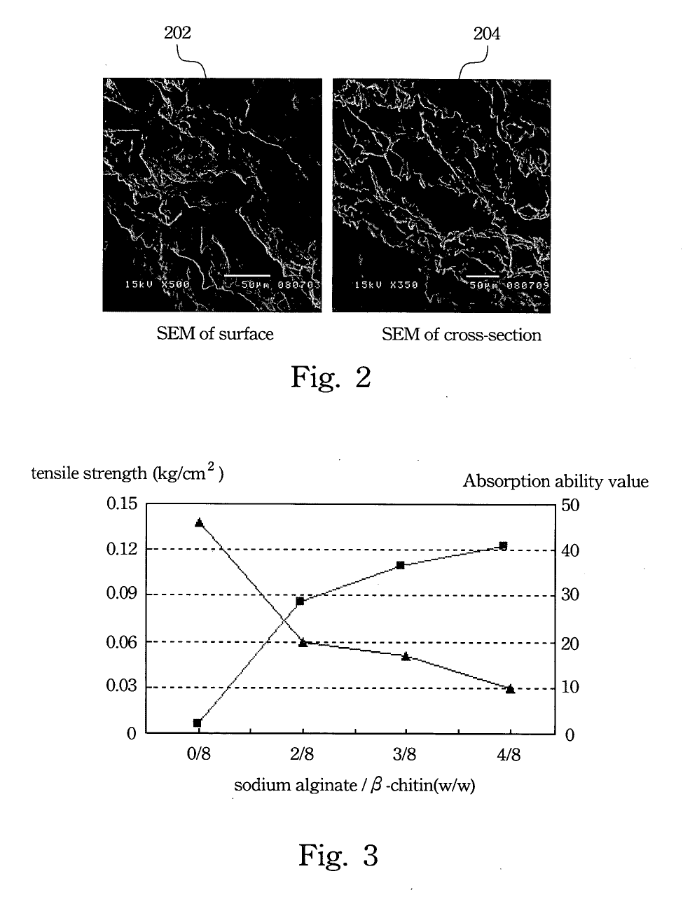

The surface and the cross-sectional structure of the wound dressing are shown in FIG. 2. The SEM pictures 202 and 204 show that chitin short staples of the wound dressing are arranged randomly and the alginate is formed as films. So the wound dressing is porous for excellent gas permeation.

### The preferred embodiment

The wound dressing of the present invention is made of chitin and alginates, in which the ratio of alginate to chitin is, for example, between about 1:9 and 9:1 (w:w). The preferred embodiment of the present invention illustrates the relationship between the ratio of alginate to chitin and the tensile strength and the absorption ability of the wound dressing.

Tensile strength and absorption ability tests

In the preferred embodiment, sodium alginate was chosen for a raw material of the present invention. Four wound dressings were made in different ratios of sodium alginate to, β-chitin, 0:8, 2:8, 3:8 and 4:8, by the method of the present invention described above. Then, the four wound dressings were tested for tensile strength by a universal testing machine, model No.HT-8504.

First, each of the four wound dressings with different ratios of alginate and β-chitin was cut into 2 cm×5 cm pieces and pulled at a speed of 50 mm/min for testing the tensile strength (dry) thereof. Another piece of the same size was immersed in saline water for 3 min, then removed from the saline bath, and excess water removed therefrom with paper. The wet wound dressings were pulled at a speed of 50 mm/min for testing the tensile strength (wet) thereof. The results are shown in Table 1.

Table 1 shows that the wound dressing with the ratio 0:8 has the worst tensile strength, whether it is wet (0.006 kg/cm²) or dry (0.046 kg/cm²). After mixing with alginate, the tensile strength is increased. For example, the tensile strength of the wet wound dressing with a ratio 2:8 is 0.086 kg/cm², over 10 times that of the wet wound dressing with a ratio 0:8. Therefore, the tensile strength of the wound dressing of the present invention increases with the ratio of alginate to chitin.

TABLE 1

| | dry sponge | | wet sponge | |
|---|---|---|---|---|
| | | Tensile | Loading | Tensile |
| Sodium alginate:β-chitin | Loading | strength | weight | strength |
| (w:w) | weight (kg) | (kg/cm²) | (kg) | (kg/cm²) |
| Sodium alginate:β-chitin | 0.46 | 0.046 | 0.06 | 0.006 |
| (0:8) | | | | |
| Sodium alginate:β-chitin | 1.23 | 0.123 | 0.86 | 0.086 |
| (2:8) | | | | |
| Sodium alginate:β-chitin | 1.74 | 0.174 | 1.10 | 0.110 |
| (3:8) | | | | |
| Sodium alginate:β-chitin | 2.30 | 0.230 | 1.23 | 0.123 |
| (4:8) | | | | |

The four wound dressings were tested for absorption ability by following steps. First, the four wound dressings with different ratio of alginate and β-chitin were cut into 2 cm×5 cm pieces and the dry weight thereof separately obtained. Then, the wound dressings were immersed in saline water for 3 minutes. After wiping off excess water on the surface thereof, each wound dressing was weighed to obtain the wet weight thereof. Then, the absorption ability was calculated by the following equation:
Absorption ability=(Wet weight−Dry weight)+(Dry weight)

As a result, table 2 shows that the absorption ability of the present invention decreases by increasing the amount of sodium alginate used in the sponge.

TABLE 2

| Sodium alginate:β-chitin | | | Absorption |
|---|---|---|---|
| (w:w) | Dry weight (g) | Wet weight (g) | ability |
| Sodium alginate:β-chitin | 0.101 | 4.756 | 46 |
| (0:8) | | | |
| Sodium alginate:β-chitin | 0.122 | 2.567 | 20 |
| (2:8) | | | |
| Sodium alginate:β-chitin | 0.110 | 1.873 | 17 |
| (3:8) | | | |
| Sodium alginate:β-chitin | 0.070 | 0.778 | 10 |
| (4:8) | | | |

The results in table 1 and in table 2 can be plotted on the curves given in FIG. 3. FIG. 3 shows the relationship of the tensile strength and the absorption ability of the wound dressings with different ratios of sodium alginate to chitin. According to FIG. 3, the tensile strength increases when the ratio of alginate to chitin increases. In contrast, the absorption ability decreases when the ratio of alginate to chitin increases. The ratio of alginate to chitin can be between 1:9 and 9:1 (w:w). In the preferred embodiment, a preferred ratio of alginate to chitin can be between 1:4 and 1:2 for better absorption and tensile strength.

Toxicity test

Wound dressings of the present invention may be applied to wounds of human or animals. Therefore, wound dressings of the present invention should be tested for toxicity. The wound dressing in a ratio of alginate to chitin 3:8 (w:w) was selected for a toxicity test. According to the specification of ASTM F813-83 for processing cell toxicity test of an implant, L929 mouse fiberblast was incubated in a 6-cm culture dish at 37° C. until cells were confluent in a monolayer. Then, the sterile wound dressing of the present invention was put in the dish in direct contact with the cells and cultured at 37° C. in a CO₂ incubator.

After 24 hours, the cells were observed by microscope and stained with 2% crystal violet to determine the cell viability. In the preferred embodiment, compared with the negative control, cells in contact with the wound dressing of the present invention lived and proliferated. Therefore, the wound dressing of the present invention is non-toxic.

Wound-repairing test

In the preferred embodiment, several samples used herein as control groups and the wound dressings of the present invention are as test groups, which are described as below:

| | Preparation method |
|---|---|
| Control group | |
| Kaltostat | Commercial product (Britcair Co.) |
| Alginate wound | 100 ml of 1% sodium alginate solution was prepared. |
| dressing made by | Polyethyleneimine and Carbodiimide were added to the |
| cross-linking method | solution in a concentration of 7.6 mM and 15 mM, |
| | respectively. After mixing and reacting for 2 hours, the |
| | solution was dried to form a sponge by freeze-drying. |
| | Then, the sponge was immersed in a 1% CaCl₂ solution to |
| | obtain a wound dressing. |
| Alginate non-woven | Calcium alginate fibers were obtained using a 5% |
| wound dressing | sodium alginate solution as a spinning solution and 5% |
| | calcium chloride solution as a coagulation-bath by wet |
| | spinning technique. The fibers were cut into short staples |
| | to form a non-woven fabric in 150 g/m². |
| Test group | |
| Alginate/chitin sponge of | Sodium alginate and β-chitin were mixed in a ratio of |
| the present invention | 3:8 to form a solution. The solution was stirred at the |
| (alginate:chitin = 3:8) | speed of 12000 rpm to transfer the β-chitin into short |
| | staples. After freeze-drying, the solution formed a |
| | sponge. The sponge was immersed in a 5% calcium |
| | chloride solution for 20 minutes, and then washed with |
| | water to remove excess salts. After the sponge was |
| | rinsed with D.I water and vacuum-dried, the alginate/chitin |
| | wound dressing of the present invention was obtained. |

The three control samples and the wound dressing of the present invention were tested for wound repairing according to the following description. Four SD-rats (male Spaque-Dawley rats with individual weights of 250 mg-300 mg) were provided as experimental subjects. After anesthetization with ether, the rats were shaved and disinfected with 10% aqueous Betadine and 70% ethanol. The rats were then cut with a surgical knife to form a wound with an area of 3 cm×3 cm and a depth down to the panniculus carnosis. After operation, the four wound dressings were cut in 3 cm×3 cm squares, and each wound dressing covered the wounds separately. Then, a supporter (such as Teggaderm (3M)) was provided to cover the wound dressings and was fixed with a self-adherence elastic bandage (3M) as a protection dressing. The supporter was, for example, a film made of PP, PE, PET, Nylon, or non-woven and fabrics made of cotton or wool, or other natural or synthetic fabrics, and the protection dressing was, for example, a PU film, bandage, non-woven, fabric, paper or tape.

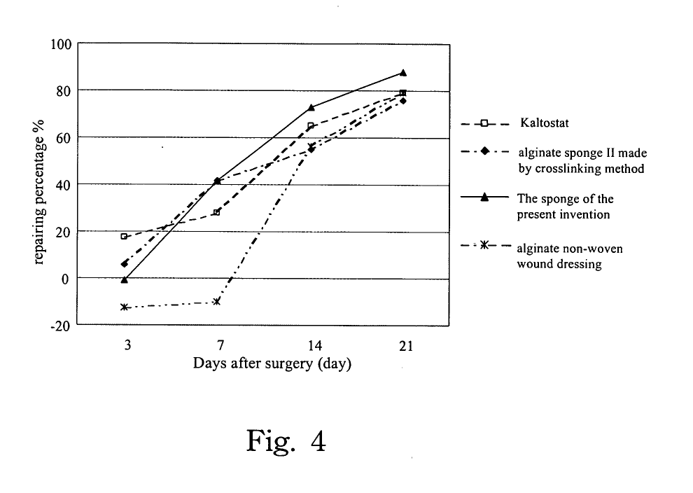

The rats were fed ad libitum and kept individually. The wound on the rats were observed on the third, the seventh, the fourteenth and the twenty-first day after operation. Each group of the test was repeated 3 times with different rats. The results are shown in FIG. 4. According to FIG. 4, the wound dressing of the present invention provides a more steady and better healing of wounds than do other wound dressings.

### CONCLUSION

The present invention advantageously promotes hemostasis, exudates absorption and gas permeation by the combination of alginate and chitin. The porous structure of the alginate is gas permeable. If the alginate of the wound dressing is calcium alginate, the wound dressing of the invention further provides hemostasis for a bleeding wound. The chitin used in the wound dressing of the present invention can adsorb the exudates, and protect the wound from infection by the antibacterial activity of chitin. The chitin also avoids release of excess calcium ions from the alginate if calcium alginate is used, and a better wound healing can be obtained by the control of calcium ion release.

Another advantage of the present invention is that the ratio of alginate to chitin can be modified according to the needs of different wounds. The ratio of chitin may be increased for additional adsorption for use on a wound with excessive exudates. The ratio of alginate can be increased for providing proper tensile strength, good hemostasis effect and convenient use. The ratio of alginate to chitin is, for example, between about 1:9 and 9:1 (w:w), and preferably between about 1:4 and 1:2.

Still another advantage of the present invention is that it is non-toxic for human and animals.

Yet another advantage of the present invention is that the wound dressing of the present invention provides a better wound healing effect and repairs a wound more steadily than other wound dressings.

According to the foregoing advantages, the wound dressing of the present invention is gas permeable, absorbs exudates, is anti-microbial, and promotes hemostasis and healing. Moreover, the wound dressing of the present invention provides proper tensile strength for operation.

The specific parameters cited in the foregoing embodiments, such as “temperature”, “concentration” and “time” are used merely for the purpose of representation, which shall not be used to restrict the scope of the present invention. Therefore, the spirit and scope of the appended claims should no be limited to the description of the preferred embodiments contained herein.

It will be apparent to those skilled in the art that various modifications and variations can be made to the method and the wound dressing of the present invention without departing from the scope or spirit of the invention. In view of the foregoing, it is intended that the present invention cover modifications and variations of this invention provided they fall within the scope of the following claims and their equivalents.

1. A wound dressing comprising chitin and alginate, wherein the alginate and the chitin are mixed in a ratio of between about 1:9 and about 9:1.

2. The wound dressing of claim 1, wherein the chitin comprises β-chitin.

3. The wound dressing of claim 2, wherein the β-chitin comprises a short staple β-chitin.

4. The wound dressing of claim 1, wherein the alginate is formed with an alkali metal alginate and an alkaline earth metal alginate.

5. The wound dressing of claim 4, wherein the alkali metal alginate is sodium alginate, potassium alginate or any combination thereof.

6. The wound dressing of claim 4, wherein the alkaline earth metal alginate is selected from a group consisting of beryllium alginate, magnesium alginate, calcium alginate, strontium alginate, barium alginate and any combination thereof.

7. The wound dressing of claim 1, wherein the ratio is between about 1:4 and about 1:2.

8. The wound dressing of claim 1, further comprising a supporter, wherein the supporter is selected from a group consisting of fabrics, non-woven and films made of PE, PU, PET, Nylon, PU, cotton and wool.

9. The wound dressing of claim 1, further comprising a protection dressing, wherein the protection dressing is selected from a group consisting of bandage, non-woven, fabric, paper, PU film and tape.

10. A method for producing a wound dressing, comprising steps of:
mixing alkali metal alginate and chitin in a ratio of between about 1:9 and about 9:1 to form a hydrogel;
forming a sponge by drying the hydrogel; and
immersing the sponge in an alkaline earth metal salt solution for forming a wound dressing.

11. The method of claim 10, wherein the alkali metal alginate is sodium alginate, potassium alginate or any combination thereof.

12. The method of claim 10, wherein the drying step comprises freeze-drying.

13. The method of claim 10, wherein the chitin comprises β-chitin.

14. The method of claim 10, further comprises a step of blending the hydrogel at high speed to form a short staple β-chitin between the steps of mixing and drying.

15. The method of claim 10, wherein the ratio is between about 1:4 and about 1:2.

16. The method of claim 10, wherein the alkaline earth metal salt is selected from a group consisting of beryllium salt, magnesium salt, calcium salt, strontium salt, barium salt and any combination thereof.

17. The method of claim 10, wherein the immersion step further comprises a step for cleaning the sponge with water or deionized water.
